(19) United States
(12) Patent Application Publication
(10) Pub. No.: US 2018/0280295 A1
(43) Pub. Date: Oct. 4, 2018

(54) SINGLE VIAL READY TO USE CABAZITAXEL FORMULATIONS WITH INCREASED STABILITY AND METHODS OF PREPARATIONS

(71) Applicants: SONG LIN, WALLINGFORD, CT (US); INDU JAVERI, WILMINGTON, MA (US); KALIAPPANADAR NELLAJAPPAN, WILMINGTON, MA (US)

(72) Inventors: SONG LIN, WALLINGFORD, CT (US); INDU JAVERI, WILMINGTON, MA (US); KALIAPPANADAR NELLAJAPPAN, WILMINGTON, MA (US)

(21) Appl. No.: 15/068,641

(22) Filed: Mar. 14, 2016

(51) Int. Cl. A61K 9/08; A61K 31/337; A61K 47/26; A61K 47/10; A61K 47/12; A61K 47/22; A61K 9/00

(52) U.S. Cl. CPC A61K 9/08; A61K 31/337; A61K 47/26; A61K 9/0019; A61K 47/12; A61K 47/22; A61K 47/10

(57) ABSTRACT

The present inventions relate to pharmaceutical formulations and formulation process comprising cabazitaxel in a single vial ready to use configuration with increased stability. The stable Cabazitaxel is ready to be added directly to the infusion solution, without any need for preparing premix solutions.

### FIELD OF THE INVENTION

The present invention relates to pharmaceutical formulations comprising cabazitaxel or its derivative. These cabazitaxel formulations are in the form of single vial configuration ready-to-use solutions or concentrates and are ready for direct dilution with infusion solution such as 0.9% sodium chloride or 5% D5W, without any need for the preparation of premix solutions.

### BACKGROUND OF THE INVENTION

The chemical name of “cabazitaxel” is (2α,5β,7β,10β, 13a)-4-acetoxy-13-({(2R,3S)-3[(tertbutoxycarbonyl)amino]-2-hydroxy-3-phenylpropanoyl}oxy)-1-hydroxy-7,10-dimethoxy-9-oxo-5,20-epoxytax-11-en-2-yl benzoate and its structural formula is:

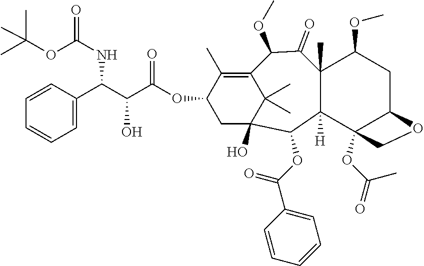 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

The empirical formula of Cabazitaxel is C₄₅H₅₇NO₁ and its molecular weight is 835.93. Cabazitaxel is a white to off-white powder. It is lipophilic, practically insoluble in water, having solubility about 8 pg per ml, and is soluble in alcohol.

Cabazitaxel is a member of the taxane family, which is the 7, 10-dimethoxy analogue of docetaxel. It is a microtubule inhibitor, which binds to tubulin and promotes its assembly into micro tubules while simultaneously inhibiting disassembly. This leads to the stabilization of microtubules, which results in the inhibition of mitotic and interphase cellular functions. It is indicated in combination with prednisone for treatment of patients with hormone-refractory metastatic prostate cancer previously treated with a docetaxel-containing treatment regimen.

Cabazitaxel is the active ingredient in products sold as under the trade name JEVTANA® by Sanofi Aventis as a sterile, non-pyrogenic, clear yellow to brownish-yellow viscous solution in single-use vials containing 60 mg cabazitaxel (calculated on an anhydrous and solvent free basis) and 1.56 g of polysorbate 80. Each ml of the cabazitaxel solution contains 40 mg cabazitaxel (anhydrous) and 1.04 g polysorbate 80.

The cabazitaxel injection consists of cabazitaxel solubilized in Tween 80 which requires two dilutions prior to intravenous infusion. The first dilution requires premixing 1.5 ml of cabazitaxel in Tween 80 with approximately 5.7 ml of clear, colorless, sterile, and non-pyrogenic solution containing 13% (w/w) ethanol in water for injection. Such pre-mix solution is supersaturated by about 400% and is susceptible to precipitation of cabazitaxel from the solution. It also requires repeated inversions for at least 45 seconds to assure complete mixing of the concentrated drug solution and the diluent. The pre-mix solution, having a concentration of 10 mg of cabazitaxel per ml should be used immediately, preferably within 30 minutes and requires further dilution before administration. A volume of premix solution calculated based on a dose of 25 mg/m² is withdrawn and injected into a PVC-free container of either 0.9% sodium chloride solution or 5% dextrose solution for infusion: After this second dilution, concentrations of cabazitaxel in the infusion solution should be between 0.10 mg/ml and 0.26 mg/ml. Diluted infusion solution should be used for intravenous administration within 8 hours, if stored at room temperature, or within 24 hours, if stored under refrigerated conditions. It is noted that the infusion period would requires about one hour. As both the pre-mix solution and the second diluted infusion solution are supersaturated, cabazitaxel may crystallize from the solutions over time. The prescribing information for cabazitaxel injection instructs that if crystals and/or particulates appear in the diluted infusion solution, it must not be used and should be discarded.

One of the difficulties with the commercially available cabazitaxel injection formulation is that the administration process is complex and involves many steps. As described above, the person administering the drug must first create a premix solution and the subsequently transfer that premix solution into an infusion bag. As cabazitaxel is extremely toxic, strict precautions should be taken in order to minimize handling hazards involving dilution of JEVTANA Injection and subsequent preparation of an infusion solution. In making the premix solution, the medical practitioner must manually invert the vial for 45 seconds repeatedly. The prescribing information for JEVTANA gives very clear instructions not to shake the vial to avoid foaming or spillage. Foaming may result in potency loss. A further difficulty of the JEVTANA product is that the premix solution must be added to the infusion bag within 30 minutes of making such admixture. Further, once added to the infusion bag, it has a limited stability in the infusion bag.

U.S. Pat. No. 5,438,072 discloses a two-part injectable composition, which involves preparing a premix solution by mixing the stock solution comprising taxane and surface active agent with a first diluent solution, and then adding this premix solution to infusion bag containing aqueous infusion solution. The first diluent solution contains an additive, which promotes the dissolution of taxane in the aqueous infusion solution in hospitals.

U.S. Pat. Nos. 5,403,858; 5,698,582; 5,714,512; 5,750,561 are directed towards substantially reducing the concentration or completely removing cremophor and ethanol from the perfusion fluid as a way to reduce the chances of probable anaphylactic manifestations. These inventions therefore consist of producing an intermediate premix solution and then adding premix solution to the infusion bag. These formulations contain taxane derivative dissolved in a surfactant selected from polysorbate or polyethoxylated castor oil, which are essentially free of ethanol, i.e., less than 5% by volume of ethanol. As described in the specification. The solutions of paclitaxel or docetaxel without ethanol are physically stable from 8 hours to several months. Furthermore, as observed, the stability of an aqueous solution of cabazitaxel in ethanol and polysorbate 80 is only eight hours when stored between 2° C. and 8° C. or at room temperature.

U.S. Patent Application Publication No. 2009/0 8354 discloses a liquid pharmaceutical formulation for parenteral administration comprising docetaxel or a pharmaceutically acceptable salt thereof, one or more glycols, and a pharmaceutically acceptable non-aqueous solvent system, wherein the formulation has a pH in the range of 2.5 to 7.

U.S. Patent Application Publication No. 2011/0130446 discloses pharmaceutical compositions for parenteral administration, comprising a taxane, at least one surfactant, at least one alkyl ester of citric acid, and ethanol in an amount less than 15% by weight.

U.S. Patent Application Publication Nos. 2010/0305202 and 2010/0267817 disclose lyophilized pharmaceutical composition comprising a water-insoluble taxoid, a cyclodextrin, and at least one hydrophilic polymer selected from the group consisting of hydroxypropylmethyl cellulose (HPMC), polyethylene glycol (PEG), and polyvinylpyrrolidone (PVP). International Application Publication No WO 2010/023321 discloses liquid pharmaceutical formulations comprising: (a) a taxane derivative or a pharmaceutically acceptable salt thereof, (b) a solvent consisting in the mixture of a pharmaceutically acceptable alcohol, a pharmaceutically acceptable polyethoxylated fatty acid ester and 30-50% by weight of water based on the total weight of the solvent. International Application Publication No WO 2009/1 5655 discloses solvates, hetero solvates, and hydrates of dimethoxydocetaxel.

There remains a need for improved injectable cabazitaxel formulations which are stable and easy to use.

### BRIEF SUMMARY OF THE INVENTION

One aspect of the present invention is directed to a stable pharmaceutical formulation for parenteral administration, comprising (i) from about 1 mg/ml to about 150 mg/ml of cabazitaxel (ii) from about 50 mg/ml to about 800 mg/ml of at least one micellar surfactant, (iii) from about 0.05 mg/ml to about 20 mg/ml of at least one stabilizer, and (iv) at least one organic solvent in a single vial configuration.

Another aspect of the present invention is directed to a method of treating cancer, comprising administering the pharmaceutical formulation which comprises (i) from about 1 mg/ml to about 150 mg/ml of cabazitaxel (ii) from about 50 mg/ml to about 800 mg/ml of at least one micellar surfactant, (iii) from about 0.05 mg/ml to about 20 mg/ml of at least one stabilizer, and (iv) at least one organic solvent in a single vial configuration.

Another aspect of the present invention is directed to a pharmaceutical formulation which comprises (i) about 1-40 mg/ml of cabazitaxel, (ii) about 100 mg/ml to about 900 mg/ml of polysorbate 80, (iii) about 0.01 mM to about 30 mM of succinic acid and (iv) about 10-90% ethanol.

A further aspect of the present invention is directed to a pharmaceutical formulation which comprises (i) 26.7 mg/ml of cabazitaxel, (ii) 710.22 mg/ml of polysorbate 80, (iii) 20 mM of succinic acid and (iv) about 33% v/v ethanol.

The contents of the patents and publications cited herein and the contents of documents cited in these patents and publications are hereby incorporated herein by reference to the extent permitted.

### BRIEF DESCRIPTION OF THE DRAWINGS

FIG. 1. Effect of time and temperature on the stability of Cabazitaxel at 4° C. (time in days vs. % impurity)

FIG. 2: Effect of time and temperature on the stability of Cabazitaxel at 40° C. (time in days vs. % impurity)

FIG. 3: Effect of time and temperature on the stability of Cabazitaxel at 60° C. (time in days vs. % impurity)

### DETAILED DESCRIPTION

As used herein, the term “cabazitaxel” includes the compound cabazitaxel, pharmaceutically acceptable salts of cabazitaxel, isomers, solvates, complexes and hydrates, anhydrous forms thereof, and any polymorphic or amorphous forms or combinations thereof.

The term “stable pharmaceutical formulation(s)” refers to any preparation of cabazitaxel having sufficient stability to allow storage at a convenient temperature, such as between about 0° C. and about 60° C., for a commercially reasonable period of time, such as at least about one week, at least about one month, at least about three months, at least about six months, at least about one year, or at least about 2 years.

The term “user(s)” refers to any qualified physician, nurse, or other practitioner who are experienced in the use of antineoplastic medicinal products.

The term “stabilizer(s)” refers to one or more components of cabazitaxel formulation that reduces (as compared with control) formation of impurity of cabazitaxel for a period of at least 12 hours, including at least 24 hours, including at least 48 hours, including at least 7 days, including at least 1 month, including at least 2 months, including at least 4 months, including at least 6 months, and including at least 9 months, 12 months, 18 months, 24 months, 36 months.

The term “micellar surfactant(s)” or “micelle-forming surfactant” refers to an amphiphilic material that spontaneously and reversibly forms a water soluble aggregate

As used herein, the term “dicarboxylic acid(s)” refers to any organic compound having at least two carboxylic acid (—COOH) functionalities therein. Some preferred examples of dicarboxylic acids that may be used in the presently described process are succinic acid, maleic acid, phthalic acid, citric acid and fumaric acid. One example of a preferable dicarboxylic acid is succinic acid.

“Reducing agent(s)” includes, but is not limited to suitable antioxidants which can be used in pharmaceutical formulation, such as citric acid, ascorbic acid, gentisic acid, glutathione, cysteine, tocopherol-derived compounds, butylated hydroxyanisole, butylated hydroxytoluene, reducing sugars such as dextrose and the like.

“Elevated temperature” means the temperature is from about 15° C. to about 80° C.

In a preferred embodiment, the micellar surfactant in the stable pharmaceutical formulation for parenteral administration is polysorbate 80, which is preferably from about 80 mg/ml to about 700 mg/ml, more preferably from about 100 mg/ml to about 600 mg/ml and the most preferably from 120 mg/ml to 500 mg/ml.

In another preferred embodiment, the organic solvent is ethanol, propanol or isopropanol.

In another preferred embodiment, the stabilizer is a reducing agent and/or a dicarboxylic acid. Preferably the dicarboxylic acid is succinic acid.

Without intending to be bound by any particular theory of operation, it is believed that the presence of a stabilizer, such succinic acid in the cabazitaxel formulation, prevents degradation of cabazitaxel or reduces the formation of all impurities. Preferably, the stabilizer, such as succinic acid, is from about 1 mg/ml to about 15 mg/ml, more preferably from about 2 mg/ml to about 10 mg/ml and the most preferably from about 3 mg/ml to about 8 mg/ml.

In another preferred embodiment, the reducing agent comprises ascorbic acid.

In a further embodiment, the sterile filtering step in the process of making the cabazitaxel formulation is performed at from about 25° C. to about 45° C., preferably at about 40° C. and more preferably at about 40° C. using 0.2μ PVDF membrane filter.

An important utility of the present invention is that it is easy to administer to patients and the formulation is physically and chemically stable.

The following examples are given as specific illustrations of the invention. It should be understood, however, that the invention is not limited to the specific details set forth in the examples. All parts and percentages in the examples, as well as in the remainder of the specification, are by weight unless otherwise specified.

Further, any range of numbers recited in the specification or paragraphs hereinafter describing or claiming various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. The term “about” when used as a modifier for, or in conjunction with, a variable, is intended to convey that the numbers and ranges disclosed herein are flexible and that practice of the present invention by those skilled in the art using temperatures, concentrations, amounts, contents, carbon numbers, and properties that are outside of the range or different from a single value, will achieve the desired result, namely, a stable pharmaceutical formulation containing cabazitaxel.

### Examples 1-8. Preparation of the Cabazitaxel Formulations

Commercial cabazitaxel formulation requires two dilutions before administration to patients. Initial dilution is to use the accompanying diluent to achieve a concentration of 10 mg/ml of cabazitaxel. To achieve the same formulation in one vial configuration, one ml of Tween-80 formulation can be diluted with 3 ml of 13% ethanol. As the density of tween 80 is 1.064, each 10 mg Cabazitaxel will require 266 mg Tween-80. To prepare 13% W/V ethanol, each ml requires 165 μL of ethanol and 835 μL water. Water is avoided as it is known to degrade cabazitaxel. Tween-80 and the 13% ethanol diluents are in the ratio of 1:3 to achieve 10 mg/ml of Cabazitaxel, each 266 mg Tween-80 was mixed with 124 μL of ethanol containing 10 mg cabazitaxel with and without stabilizers.

Cabazitaxel formulations containing tricarboxylic acids, such as citric acid and tartaric acid in presence of PEG as described in WO 2013024495 A1 and CN102068407A were also prepared as side-by-side comparison to the claimed formulations.

The pharmaceutical formulations of cabazitaxel was made by preparing a solution of cabazitaxel in the required amount of an organic solvent in the presence of a stabilizer, followed by adding a micellar surfactant, with or without stirring. The stabilizer was either a dicarboxylic acid, or a reducing agent.

The resulting formulation was a clear solution with limited foaming. To maintain the sterility of the formulation, it was sterile filtered using a 0.2μ filter. The cabazitaxel formulations prepared as such were packaged in suitable container and stored at 4° C., 40° C. and 60° C. over three months. After storage, stability of the formulations was tested using HPLC analysis. The pharmaceutical formulations typically complied with the International Conference on Harmonization (ICH) Guidelines.

Cabazitaxel and excipients were solubilized in ethanol. Initially stock solutions containing stabilizers were prepared at 6, 15 and 30 mM concentration in ethanol to achieve the final concentration of 2, 5 and 10 mM concentration of stabilizer. Dicarboxylic acid such as succinic acid, aspartic acid and glutamic acids were assessed. Tricarboxylic acid was used as control. In addition, the effects of reducing agents like ascorbic acid and boric acids were also assessed.

While solubilizing the excipient in ethanol, it was noted that aspartic acid, glutamic acid and boric acid were not soluble in ethanol even at 6 mM concentration. These excipients were therefore not used as stabilizer. However, ascorbic acid at 6 mM was evaluated as a stabilizer (15 mM ascorbic acid is not soluble in ethanol). Cabazitaxel with and without excipients were dissolved in ethanol or ethanol containing excipients.

About 266 mg Tween-80 was weighed into a 20 ml flint vial and mixed with 124 μL ethanol or ethanol containing excipients such as succinic acid, ascorbic acid or citric acid, with the addition of 10 mg cabazitaxel. 100 μL of the formulation was aliquoted in 1 ml HPLC vial and screw capped and kept at 4° C., 40° C. and 60° C. The 20 ml vials were stoppered and crimped. Ten μL of the sample were diluted with 257 μL of ethanol to achieve at 1 mg/ml concentration and the stability of the formulations was assessed by RP-HPLC at different timings. Tween-80 and ethanol was used as placebo. The concentration of cabazitaxel in the formulation is 26.7 mg/ml. The stabilizers and their amounts used in examples 1-8 are listed below:

Example 1. Tween 80 and ethanol—negative control

Example 2. 6 mM Succinic acid

Example 3. 15 mM Succinic acid

Example 4. 30 mM Succinic acid

Example 5. 6 mM Ascorbic acid

Example 6. 6 mM citric acid

Example 7. 15 mM citric acid

Example 8. 30 mM citric acid.

The data on the chromatogram of the placebo indicated that Tween-80 and ethanol have some minor peaks, which can be seen only after zooming. The % impurity of cabazitaxel in different excipients/stabilizers kept at different temperature and time were presented in Tables 1-3. The data indicated that the total impurity is highest for the formulation containing citrate when kept at 60° C. for 7 days. The least impurity is seen for the formulation containing 6 mM ascorbic acid. Less impurity is seen for the formulation containing succinic acid when compared to formulation containing citric acid. It is interesting to note though citric acid reduced the formation of impurity which eluted at 9.48 minutes. However, it promoted the impurities which eluted at 12, 17.84, 20.1 and 22.9 minutes as compared with succinic acid or ascorbic acid. The formulation containing ascorbic acid showed the least impurities with the retention time 10-13.5 minutes and impurities which eluted after the major peaks.

TABLE 1
Effect of time and temperature on the stability of cabazitaxel: % Impurity: 4° C.

| | | 4° C. | 4° C. | 4° C. | 4° C. | 4° C. | 4° C. | 4° C. |
|---|---|---|---|---|---|---|---|---|
| Examples | Formulations | 24 hr | 48 hr | 72 hr | 5 days | 6 days | 9 days | 10 days |
| 1 | control | 1.33 | | 1.35 | | 1.19 | | 1.33 |
| 2 | 6 mM Suc | 1.19 | | 1.21 | | 1.12 | | 1.21 |
| 3 | 15 mM Suc | 1.29 | | 1.25 | | 1.29 | | 1.15 |
| 4 | 30 mM Suc | 1.24 | | 1.21 | | 1.15 | | 1.27 |
| 5 | 6 mM Asc | 1.29 | | 1.37 | | 1.18 | | 1.49 |
| 6 | 6 mM Cit | 1.23 | 1.18 | | 1.10 | | 1.29 | |
| 7 | 15 mM Cit | 1.32 | 1.21 | | 1.28 | | 1.29 | |
| 8 | 30 mM Cit | 1.38 | 1.31 | | 1.23 | | 1.24 | |

TABLE 2
Effect of time and temperature on the stability of Cabazitaxel: % Impurity: 40° C.

| | | 40° C. | 40° C. | 40° C. | 40° C. | 40° C. | 40° C. | 40° C. |
|---|---|---|---|---|---|---|---|---|
| Examples | Formulations | 24 hr | 48 hr | 72 hr | 5 days | 6 days | 9 days | 10 days |
| 1 | control | 1.35 | | 1.44 | | 1.45 | | 1.55 |
| 2 | 6 mM Suc | 1.32 | | 1.37 | | 1.36 | | 1.46 |
| 3 | 15 mM Suc | 1.31 | | 1.27 | | 1.35 | | 1.58 |
| 4 | 30 mM Suc | 1.27 | | 1.31 | | 1.29 | | 1.40 |
| 5 | 6 mM Asc | 1.46 | | 1.40 | | 1.49 | | 1.75 |
| 6 | 6 mM Cit | | 1.32 | | 1.30 | | 1.38 | |
| 7 | 15 mM Cit | | 1.29 | | 1.23 | | 1.38 | |
| 8 | 30 mM Cit | | 1.30 | | 1.24 | | 1.21 | |

TABLE 3
Effect of time and temperature on the stability of Cabazitaxel: % Impurity: 60° C.

| | | | 40° C. 48 hrs | 40° C. 72 hrs | 40° C. 48 hrs | 40° C. 72 hrs |
|---|---|---|---|---|---|---|
| | | | and 60° C. | and 60° C. | and 60° C. | and 60° C. |
| Examples | Formulations | | 3 days | 3 days | 7 days | 7 days |
| 1 | control | | | 2.17 | | 3.91 |
| 2 | 6 | mM Suc | | 1.99 | | 4.09 |
| 3 | 15 | mM Suc | | 1.81 | | 3.88 |
| 4 | 30 | mM Suc | | 1.71 | | 3.38 |
| 5 | 6 | mM Asc | | 1.88 | | 2.78 |
| 6 | 6 | mM Cit | 1.61 | | 3.90 | |
| 7 | 15 | mM Cit | 2.07 | | 6.59 | |
| 8 | 30 | mM Cit | 1.69 | | 6.11 | |

### Example 9-17

In the previous examples 1-8, it was shown that succinic acid and ascorbic acid provided better stability to cabazitaxel than citric acid, when the formulation is kept at 40° C. for 3 days and 60° C. for 7 days. In developing formulation for these examples, the design was to evaluate stability of final cabazitaxel formulations containing, 5, 10, or 20 mM succinic acid, 6 or 8 mM ascorbic acid and combination of 5 mM succinic acid and 2 mM ascorbic acid and 2 mM succinic acid 2 mM ascorbic acids. However, preparation with ascorbic acid >2 mM was not feasible, as we had noted before, because the solubilization of stock ascorbic acid in ethanol requires heating at 75° C. for 20 minutes. Due to difficulties of solubilizing ascorbic acid in ethanol, the following formulations were evaluated by keeping 200 μL aliquots of sample at 4° C., 40° C. and 60° C. for 7 days and 14 days.

The stabilizers and amounts used in examples 9-17 are listed below:

Example 9. Tween-80 and ethanol—negative control

Example 10. 5 mM citric acid—positive control

Example 11. 5 mM succinic acid

Example 12. 10 mM succinic acid

Example 13. 20 mM succinic acid

Example 14. 2 mM ascorbic acid

Example 15. 2.7 mM ascorbic acid

Example 16. 5 mM succinic acid and 2 mM ascorbic acid

Example 17. 2 mM succinic acid and 2 mM ascorbic acid

About 532 mg Tween-80 was weighed in a 20 ml 20 mM flint vial and mixed with 248 μL ethanol or ethanol containing excipients containing 20 mg cabazitaxel. 200 μL of the formulations were aliquoted in 1 ml HPLC vial and screw capped and kept at 4° C., 40° C. and 60° C. Ten μL of the sample was diluted with 257 μL of ethanol to achieve at 1 mg/ml concentration and the stability of the formulations was assessed by RP-HPLC at different timings. Tween-80 and ethanol were used as placebo.

The data on the examples 1-8 indicated that citric acid reduced the formation of impurity which elutes at 9.2 minutes, when the formulation was kept at 60° C. for 7 days. However citric acid accelerated the formation of the impurities, which eluted after the major peak. It is surprising to note that succinic acid reduced the formation of impurity which elutes at 9.2 minutes, and the impurity eluting after main peak, under similar conditions. This stabilization effect was found to depend on the concentration of succinic acid.

In the examples 9-17, the data was consistent with the previous examples 1-8. The formulation containing 15 mM citric acid, at 60° C. for 14 days, reduced the formation of impurity which elutes at 9.2 minutes as compared to the formulation without citric acid. As seen previously, it accelerated the formation of impurities which eluted after the major peak. On the other hand, succinic acid reduced the formation of impurity which elutes at 9.2 minutes, and the impurity eluting after main peak, under similar conditions. This stabilization effect depended on the concentration of succinic acid. At 60 mM concentration, the least amount of impurity was seen for the peaks eluted at 9.2 minutes, and impurities which eluted after the major peak were relatively less compared to other formulations (see Table 4).

Though formulation containing ascorbic acid or in combination with succinic acid was desired with respect to stability, heating in the process was essential to achieve 6 mM concentration. In the presence of 60 mM concentration of succinic acid, the solubility of ascorbic acid is further reduced to less than 2 mM even after heating. The quantity of ascorbic acid oxidized during heating was not known. Based on the T7 and T14 data, the formulation containing 20 mM succinic acid (60 mM stock in ethanol) cabazitaxel performed better than other formulations.

TABLE 4
Effect of time and temperature on the stability of Cabazitaxel: % Impurity

| | | 4° C. | 4° C. | 40° C. | 40° C. | 60° C. | 60° C. |
|---|---|---|---|---|---|---|---|
| Examples | Formulations | T7 | T14 | T7 | T14 | T7 | T14 |
| 9 | Tw-80-EtOH control | 1.70 | 1.61 | 1.62 | 2.18 | 3.59 | 7.20 |
| 10 | 5 mM Cit | 1.64 | 1.58 | 1.56 | 1.92 | 3.63 | 6.93 |
| 11 | 5 mM Suc | 1.36 | 1.48 | 1.52 | 1.57 | 3.04 | 5.15 |
| 12 | 10 mM Suc | 1.42 | 1.58 | 1.50 | 1.58 | 3.22 | 6.21 |
| 13 | 20 mM Suc | 1.43 | 1.43 | 1.39 | 1.25 | 2.48 | 4.75 |
| 14 | 2 mM Asc | 1.52 | 1.38 | 1.22 | 1.68 | 3.47 | 5.68 |
| 15 | 2.7 mM Asc | 1.40 | 1.54 | 1.41 | 2.14 | 3.15 | 5.68 |
| 16 | 5 mM Suc 2 mM Asc | 1.35 | 1.66 | 1.46 | 2.15 | 2.96 | 5.34 |
| 17 | 2 mM Suc2 mM Asc | 1.52 | 1.54 | 1.47 | 1.09 | 3.30 | 5.79 |

### Examples 18-20

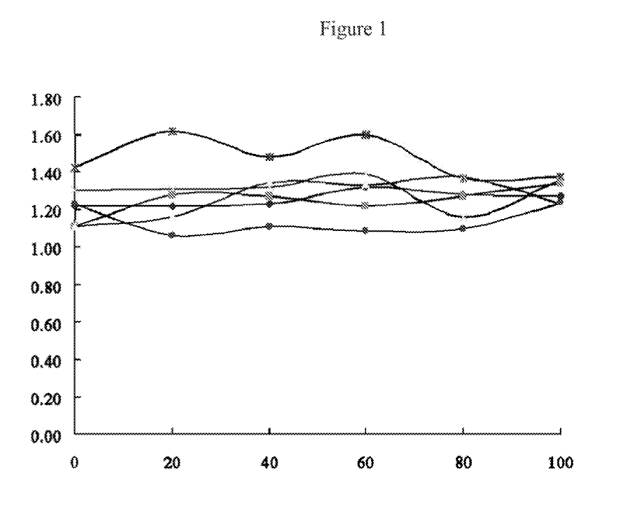

Two different batches of cabazitaxel were formulated with Tween-80-ethanol-succinic acid, or Tween-80-ethanol (negative control as detailed in the examples 9-17 or tween 80 similar to reference Jevtana (40 mg/ml) and the stability of the formulations were assessed for 90 days at 4° C., 40° C., and 60° C. respectively. Succinic acid is used in the final concentration of 20 mM. The data indicated that surprisingly cabazitaxel formulated with 20 mM succinic acid, stabilized better than cabazitaxel formulated in Tween-80 without ethanol (Jevtana) (Table 5-7 and FIGS. 1-3).

TABLE 5
Effect of time and temperature on the stability
of Cabazitaxel: % impurity: 4° C.*

| Exam- | | 4° C. | 4° C. | 4° C. | 4° C. | 4° C. | 4° C. |
|---|---|---|---|---|---|---|---|
| ples | Formulations | T7 | T14 | T21 | T30 | T60 | T90 |
| 18 | Suc-Batch 1 | 1.22 | 1.11 | 1.11 | 1.30 | 1.42 | 1.23 |
| 18 | Suc-Batch 2 | 1.22 | 1.28 | 1.16 | 1.31 | 1.62 | 1.06 |
| 19 | Tw-80-etoh Batch 1 | 1.23 | 1.27 | 1.34 | 1.32 | 1.48 | 1.11 |
| 19 | Tw-80-etoh Batch 2 | 1.32 | 1.22 | 1.33 | 1.39 | 1.60 | 1.08 |
| 20 | Tween-80 Batch 1 | 1.28 | 1.27 | 1.37 | 1.16 | 1.36 | 1.09 |
| 20 | Tween-80 Batch 2 | 1.27 | 1.34 | 1.23 | 1.36 | 1.38 | 1.23 |

*No significant increase in impurity at 4° C. (See FIG. 1)

TABLE 6
Effect of time and temperature on the stability of Cabazitaxel: % impurity: 40° C.*

| | | 40° C. | 40° C. | 40° C. | 40° C. | 40° C. | 40° C. |
|---|---|---|---|---|---|---|---|
| Examples | Formulations | T7 | T14 | T21 | 1.30 | T60 | T90 |
| 18 | Suc-Batch 1 | 1.43 | 1.52 | 1.69 | 1.81 | 2.29 | 2.39 |
| 18 | Suc-Batch 2 | 1.24 | 1.33 | 1.59 | 1.72 | 2.49 | 2.86 |
| 19 | Tw-80-etoh Batch 1 | 1.54 | 1.86 | 2.04 | 2.54 | 4.65 | 7.29 |
| 19 | Tw-80-etoh Batch 2 | 1.10 | 1.46 | 1.91 | 2.00 | 3.43 | 4.63 |
| 20 | Tween-80 Batch 1 | 1.35 | 1.68 | 1.82 | 2.31 | 5.00 | 6.10 |
| 20 | Tween-80 Batch 2 | 1.24 | 1.44 | 1.41 | 2.04 | 4.85 | 6.12 |

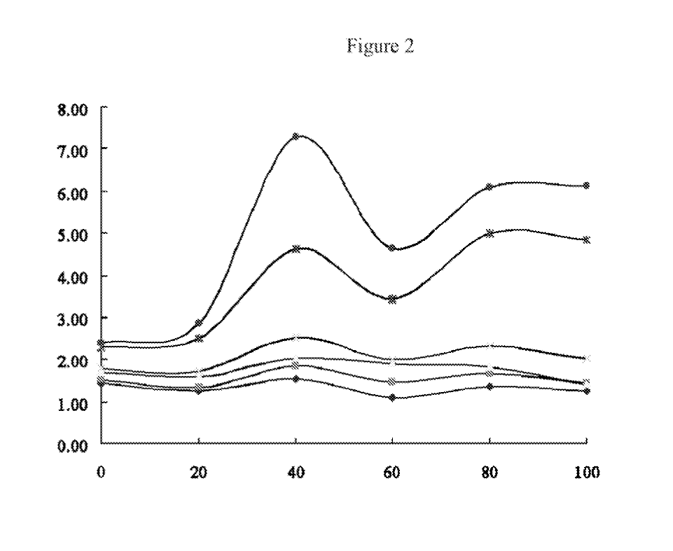

*Significant increase in impurity at 40° C. (See FIG. 2); least impurity in formulation containing succinic acid

TABLE 7
Effect of time and temperature on the stability of Cabaxitaxel: % impurity: 60° C.

| | | 60° C. | 60° C. | 60° C. | 60° C. | 60° C. | 60° C. |
|---|---|---|---|---|---|---|---|
| Examples | Formulations | T7 | T14 | T21 | T30 | T60 | T90 |
| 18 | Sac-Batch 1 | 1.62 | 2.49 | 3.76 | 5.15 | 9.7 | 13.2 |
| 18 | Sac-Batch 2 | 2.07 | 2.83 | 3.84 | 5.55 | 9.9 | 13.1 |
| 19 | Tw-80-etoh Batch 1 | 3.16 | 4.93 | 7.79 | 10.85 | 16.9 | 21.6 |
| 19 | Tw-80-etoh Batch 2 | 3.41 | 5.70 | 9.16 | 13.77 | 24.6 | 40.3 |
| 20 | Tween-80 Batch 1 | 2.51 | 3.31 | 5.00 | 6.64 | 11.5 | 13.2 |
| 20 | Tween-80 Batch 2 | 2.31 | 3.48 | 5.12 | 6.84 | 10.9 | 12.8 |

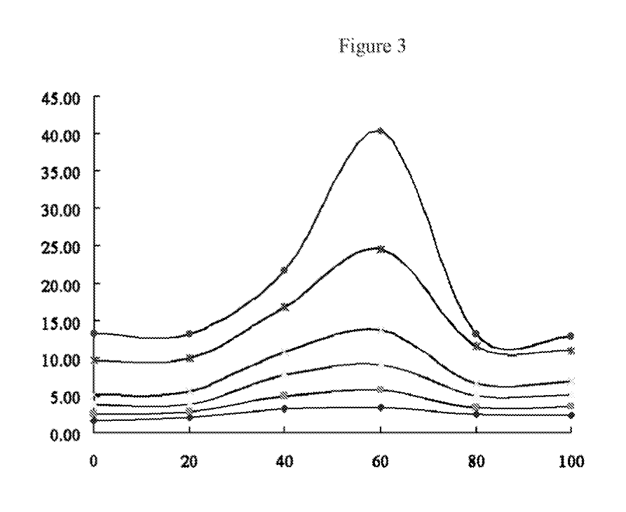

* Significant increase in impurity at 60° C. (See FIG. 3); least impurity in formulation containing succinic acid.

### Example 21: Formulation Process with 3 Grams of Cabazitaxel

A magnetic stirrer bar was transferred to a one liter screw capped Pyrex bottle. 2.6637 g Cabazitaxel and 235.6 mg of succinic acid (NF; EMD Emprove Cat#1.00681.5003) were weighed separately and transferred to the bottle. The bottle was placed on a balance (PG 5002-S) and tarred to zero. 26.06 g of ethanol (Spectrum 200 proof USP Cat # Et107) was added and removed from the balance. The content was stirred at a speed of 5 using a coming magnetic stirrer. It took 25 minutes to dissolve the cabazitaxel and succinic acid in ethanol. The bottle was transferred to the balance and tarred again. 70.77 g of Tween-80 (NOF corporation Lot #607361 A) was added and removed from the balance. The content has 2 layers, the upper ethanol layer and lower Tween-80 layers. The bottle was screw capped and the content was stirred. The mixing was completed in 5 minutes. One ml aliquot was removed and stored at 4° C. The content was kept at 40° C. for 1 hr 20 minutes so that the formulation temperature reached 38.1° C. One ml aliquot was removed and stored at 4° C. The formulation was sterile filtered using a 0.22μ PVDF stericup & steritop Millipore (150 ml funnel and 150 ml Receiver bottle). It should be noted that the formulation kept at 40° C. is stable at least for a week without any degradation with a purity of more than 99%. Filtration took 15 minutes to filter and the filtered sample was stored at 4° C. One ml aliquot was removed and stored at 4° C. All the aliquots removed during the upscale process were analyzed by RP-HPLC (Table 8). The data indicated that heating at 40° C. and filtration using PVDF membrane did not affect either the recovery or purity. When the filtration process was carried out at room temperature, the filtration rate was very slow. After filtration of 50 ml of the solution, the filter clogged completely. It was concluded that the formulation was easily scalable. Heating at 40° C. helped to make the cabazitaxel formulation easy to be filtrated, saving time and reducing loss of product. Heating at 40° C. did not affect the recovery, purity/impurity of cabazitaxel formulation

TABLE 8
In process chromatographic analysis of Cabazitaxel formulation

| | Major peak | % | % |
|---|---|---|---|
| Formulations | AUC | Purity | Impurity |
| Formulation before heat | 20311 | 98.8502 | 1.1498 |
| Formulation after heat at 40° C. and | 19148 | 99.0049 | 0.9951 |
| before filtration | | | |
| Formulation after heat, at 40° C. and | 20487 | 98.9639 | 1.0361 |
| after 0.22μ filtered | | | |

### Example 22. Three Month Stability Test

Different aliquots of brand name cabazitaxel concentrate were made in a sterile hood. The original brand name cabazitaxel concentrate was carefully removed in hood and the flip of crimp was removed after cleaning the crimp with dehydrated ethanol. As the concentrate is formulated in Tween 80, the formulation was very viscous, 18 G 1½ size was used to aliquot the concentrate. Sterile needle (B-D 18 G 1½ size) was locked on a BD 10 mL syringe with Luer-Lok™ Tip. The holobutyle rubber stopper was punctured using the syringe and approximately 1 mL of brand name cabazitaxel was removed. Care was taken not to introduce any air during this process. Approximately 100-200 μL of concentrate was distributed into a 3 mL 13 mm depyrogenated (200° C. for at least 2 hrs). Applicants expected 7 aliquots from this process. However, 6 aliquots were made by Applicants. Four aliquots were used for 25° C. stability at T0, T1 month, T2 month and T3 month stability. Two vial aliquots were used for 40° C. stability for T1 month and T2 month stability. For the third month stability at 40° C., the residual sample in the original unopened vial of brand name cabazitaxel was used. The washed and sterilized vials were closed with 13 mm washed and sterilized halo butyl rubber stopper and crimped using a flip off crimp. Based on the availability of the brand name cabazitaxel, and formulation of the present application, vials, the samples were distributed as follows for the stability studies (Table 9)

TABLE 9
Distribution of brand name cabazitaxel and new formulation for stability study
(Accelerated stability program on the stability of Inno-001 and Jevtana).

| Sample | # vials | Conditions | 0 months | 1 months | 2 months | 3 months |
|---|---|---|---|---|---|---|
| Inno-001 | 4 (20 mL | 25° C./60% | ✓ | ✓ | ✓ | ✓ |
| | vial) | RH | | | | |
| RLD | 4 (3 mL | 25° C./60% | ✓ | ✓ | ✓ | ✓ |
| | vial) | RH | | | | |
| Inno-001 | 3 (20 mL | 40° C./75% | | ✓ | ✓ | ✓ |
| | vial) | RH | | | | |
| RLD | 2 (3 mL | 40° C./75% | | ✓ | ✓ | |
| | vial) | RH | | | | |

The original Jevtana diluents were carefully removed in hood and the flip of crimp was removed after cleaning the crimp with dehydrated ethanol. Sterile needle (B-D 18 G 1½ size) was locked on a BD 10 mL syringe with Luer-Lok™ Tip. The holobuty rubber stopper was punctured using the syringe and approximately 3 mL Jevtana diluent was removed. Care was taken not to introduce any air during this process. Approximately 500 μL of diluent as distributed into a 3 mL 13 mm depyrogenated (200° C. for at least 2 hrs) vials. CuriRx expected 5 aliquots from this process. However, 7 vials were aliquoted with the Jevtana diluents. The vials were closed with washed and sterilized 13 mm halo butyl rubber stopper and crimped using a flip off crimp. The distribution of diluents vials are presented in Table 10.

TABLE 10
Distribution of Jevtana diluents vials for stability study

| | # vials | | 0 months | 1 months | 2 months | 3 months |
|---|---|---|---|---|---|---|
| RLD | 4 | 25° C./60% | 3 mL vials | 3 mL vials | 3 mL vials | 3 mL vials |
| diluents | | RH | | | | |
| RLD | 2 | 40° C./75% | | 3 mL vials | 3 mL vials | |
| diluents | | RH | | | | |

### Aliquots of New Formulation:

The pharmaceutical formulations of Cabazitaxel is made by preparing a solution of Cabazitaxel in the required amount of an organic solvent in the presence of a stabilizer, followed by adding a micellar surfactant, with stirring. The stabilizer exhibiting more stability to Cabazitaxel manufactured by Innomax (Inno-001) was a dicarboxylic acid which will not infringe the patent of the Innovator formulation. The final Inno-001 formulation contains 26.7 mg/mL Cabazitaxel, 20 mM succinic acid, 710.22 mg tween 80 and 330 μL ethanol.

The resulting formulation is a clear solution with limited foaming. To maintain the sterility of the formulation, it is sterile filtered using a 0.2μ filter. The 60 mg dose of Inno-001 formulations prepared are kept in 20 mL vials and stored at 25° C. (60% RH) and 40° C. (75% RH) for 3 months. After storage, stability of the formulations is tested using HPLC analysis.

The samples were removed at different time points and diluted to 1 mg/mL before injecting to HPLC. Thirty μL of the Inno-001 formulation diluted with 771 μL of 0.9% saline or 5% D5W, to achieve 1 mg/mL concentration before HPLC analysis. The stability of the formulations was assessed by RP-HPLC at 0 and 8 hr time point after dilution with 0.9% saline or 5% D5W.

For Jevtana, 30 μL active Cabazitaxel was initially diluted with 90 μL of the ethanol diluents. This was further diluted with 1.080 mL of 0.9% saline or 5% D5w. The stability of the formulations was assessed by RP-HPLC

The physical appearance data indicates that Jevtana is slightly yellowish and very viscous. It is not easily syringe able. However, INN001 is very clear like water and it is not viscous. It is easily syringe able. The data on stability of INN001 and Jevtana are presented in FIG. 4a and FIG. 4b. Y axis was kept at constant for a comparison. The HPLC data indicates that INN001 is more stable than commercial Jevtana based on the % Impurities formed during the stability time point. The peaks after 22.6 minutes were not integrated, as tween 80 peaks interfered especially for the Jevtana sample (Table 11 and 12).

TABLE 11
Effect of time and temperature (25° C.) on the stability of Jevtana and Inno-001:
RP-HPLC immediately after dilution in D5W or saline and after 8 hrs at room temperature

| Formulations 25° C. | AUC | Purity | Impurity | AUC t8 hr | Purity t8 hr | Impurity t8 hr |
|---|---|---|---|---|---|---|
| Inno-001T0 M D5W | 10126 | 99.89 | 0.11 | 10145 | 99.81 | 0.19 |
| Inno-001 T0 M saline | 10317 | 99.79 | 0.21 | 10358 | 99.79 | 0.21 |
| Jevtana T0 M D5W | 10979 | 99.56 | 0.44 | 11037 | 99.54 | 0.46 |
| Jevtana T0 M Saline | 9569 | 99.62 | 0.38 | 9698 | 99.62 | 0.38 |
| Inno-001 T1 M D5W | 9879 | 99.76 | 0.24 | 9877 | 99.61 | 0.39 |
| Inno-001 T1 M saline | 8835 | 99.67 | 0.33 | 8840 | 99.55 | 0.45 |
| Jevtana T1 M D5W | 20864 | 94.16 | 5.84 | 17250 | 95.22 | 4.78 |
| Jevtana T1 M Saline | 8345 | 94.92 | 5.08 | 8340 | 94.80 | 5.20 |
| Inno-001 T2 M D5W | 10175 | 99.76 | 0.24 | 10176 | 99.63 | 0.37 |
| Inno-001 T2 M saline | 9614 | 99.60 | 0.40 | 9622 | 99.62 | 0.38 |
| Jevtana T2 M D5W | 8737 | 94.77 | 5.23 | 8722 | 95.07 | 4.93 |
| Jevtana T2 M Saline | 9024 | 94.70 | 5.30 | 9002 | 94.95 | 5.05 |
| Inno-001 T3 M D5W | 9849 | 99.71 | 0.29 | 9858 | 99.67 | 0.33 |
| Inno-001 T3 M saline | 9721 | 99.64 | 0.36 | 9682 | 99.72 | 0.28 |
| Jevtana T3 M D5W | 14653 | 94.53 | 5.47 | 14644 | 94.45 | 5.55 |
| Jevtana T3 M Saline | 13236 | 93.52 | 6.48 | 13212 | 93.04 | 6.96 |

TABLE 12
Effect of time and temperature (40° C.) on the stability of Jevtana and Inno-001:
RP-HPLC immediately after dilution in D5W or saline and after 8 hrs at room temperature

| Formulations 40° C. | AUC | Purity | Impurity | AUC t8 hr | Purity t8 hr | Impurity t8 hr |
|---|---|---|---|---|---|---|
| Inno-001 T0 M D5W | 10127 | 99.79 | 0.21 | 10147 | 99.69 | 0.31 |
| Inno-001 T0 M saline | 10294 | 99.78 | 0.22 | 10321 | 99.83 | 0.17 |
| Jevtana T0 M D5W | 10979 | 99.56 | 0.44 | 11037 | 99.54 | 0.46 |
| Jevtana T0 M Saline | 9569 | 99.62 | 0.38 | 9698 | 99.62 | 0.38 |
| Inno-001 T1 M D5W | 9670 | 99.63 | 0.37 | 9709 | 99.47 | 0.53 |
| Inno-001 T1 M saline | 9251 | 99.63 | 0.37 | 9267 | 99.48 | 0.52 |
| Jevtana T1 M D5W | 8447 | 95.28 | 4.72 | 8429 | 95.33 | 4.67 |
| Jevtana T1 M Saline | 7812 | 95.29 | 4.71 | 7819 | 95.29 | 4.71 |
| Inno-001 T2 M D5W | 8959 | 99.16 | 0.84 | 10148 | 99.56 | 0.44 |
| Inno-001 T2 M saline | 9336 | 99.22 | 0.78 | 9320 | 99.16 | 0.84 |
| Jevtana T2 M D5W | 8020 | 95.19 | 4.81 | 8017 | 95.05 | 4.95 |
| Jevtana T2 M Saline | 9088 | 95.75 | 4.25 | 9042 | 95.94 | 4.06 |
| Inno-001 T3 M D5W | 9134 | 98.84 | 1.16 | 9125 | 98.93 | 1.07 |
| Inno-001 T3 M saline | 9505 | 98.90 | 1.10 | 9485 | 98.89 | 1.11 |
| Jevtana T3 M D5W | ND | ND | ND | ND | ND | ND |
| Jevtana T3 M Saline | ND | ND | ND | ND | ND | ND |

### Example 23

A comparative study of the stability of Jevtana and INN001 was conducted and the testing results are shown in Tables 13 and 14.

TABLE 13
Stability of INN001 at 25° C. for 2 months

| Inno T0 | Inno T0 | Inno T1 | Inno T1 | Inno T2 | Inno T2 |
|---|---|---|---|---|---|
| M D 5 W | M saline | M D 5W | M saline | M D5W | M saline |
| 0.11 | 0.21 | 0.24 | 0.33 | 0.24 | 0.40 |

TABLE 14
Stability of Jevtana at 25° C. for 2 months

| Jevtana T0 | Jevtana T0 | Jevtana T1 | Jevtana T1 | Jevtana T2 | Jevtana T2 |
|---|---|---|---|---|---|
| M D5W | M Saline | M D 5W | M Saline | M D5W | M Saline |
| 0.44 | 0.38 | 5.84 | 5.08 | 5.23 | 5.30 |

It was unexpectedly discovered that the stability of INN001 far exceeded that of Jevtana at 25° C. for 2 months.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in the art, without departing from the spirit of the invention.

What is claimed is:

1. A stable pharmaceutical formulation for parenteral administration, comprising (i) from about 1 mg/ml to about 150 mg/ml of cabazitaxel (ii) from about 50 mg/ml to about 800 mg/ml of at least one micellar surfactant, (iii) from about 0.05 mg/ml to about 20 mg/ml of at least one stabilizer, and (iv) at least one organic solvent in a single vial configuration.

2. The pharmaceutical formulation of claim 1, wherein said at least one micellar surfactant comprises polysorbate 80.

3. The pharmaceutical formulation of claim 1, wherein said at least one organic solvent comprises ethanol, propanol or isopropanol.

4. The pharmaceutical formulation of claim 1, wherein said at least one stabilizer comprises a reducing agent and/or a dicarboxylic acid.

5. The pharmaceutical formulation of claim 4, wherein said dicarboxylic acid is succinic acid.

6. The pharmaceutical formulation of claim 4, wherein said succinic acid reduces the formation of all impurities.

7. The pharmaceutical formulation of claim 4, wherein said reducing agent comprises ascorbic acid.

8. The pharmaceutical formulation of claim 3, wherein said polysorbate 80 is from about 80 mg/ml to about 700 mg/ml.

9. The pharmaceutical formulation of claim 8, wherein said polysorbate 80 is from about 100 mg/ml to about 600 mg/ml.

10. The pharmaceutical formulation of claim 5, wherein said succinic acid is from about 1 mg/ml to about 15 mg/ml.

11. The pharmaceutical formulation of claim 10, wherein said succinic acid is from about 2 mg % ml to about 10 mg/ml.

12. A process for scaling up the production of the formulation of claim 1, comprising the step of (i) by mixing cabazitaxel, succinic acid, ethanol and polysorbate 80 and (ii) sterile filtering the mixture from step (i) using 0.2μ filter at an elevated temperature.

13. The process of claim 12, wherein said sterile filtering in step (ii) is performed at from about 25° C. to about 45° C.

14. The process of claim 12, wherein said sterile filtering in step (ii) is performed at about 40° C.

15. The process of claim 12, wherein said sterile filtering in step (ii) is performed at about 40° C. using 0.2μ PVDF membrane filter.

16. The pharmaceutical formulation of claim 1, comprising (i) about 1-40 mg/ml of cabazitaxel, (ii) about 100 mg/ml to about 900 mg/ml of polysorbate 80, (iii) about 0.01 mM to about 30 mM of succinic acid and (iv) about 10-90% ethanol.

17. The pharmaceutical formulation of claim 1, preferably comprising (i) 26.7 mg/ml of cabazitaxel, (ii) 710.22 mg/ml of polysorbate 80, (iii) 20 mM of succinic acid and (iv) about 33% v/v ethanol.

18. The pharmaceutical formulation of claim 17 is less viscous and can be easily filled into a syringe.

19. The pharmaceutical formulation of claim 17, wherein said formulation is colorless

20. A method of treating cancer, comprising administering the pharmaceutical formulation of claim 1.
